(19) United States
(12) Patent Application Publication
(10) Pub. No.: US 2024/0358861 A1
(43) Pub. Date: Oct. 31, 2024

(54) COMPOSITIONS AND METHODS OF FLUORESCEIN SODIUM FOR EARLY DIAGNOSIS OF CERVICAL CANCER

(71) Applicant: THE MOEIN FAMILY TRUST, San Diego, CA (US)

(72) Inventors: Sudabeh MOEIN, Saratoga, CA (US); Lawrence Benjamin FISCHEL, Roseville, CA (US)

(21) Appl. No.: 18/646,181

(22) Filed: Apr. 25, 2024

Related U.S. Application Data
(60) Provisional application No. 63/498,103, filed on Apr. 25, 2023.

(51) Int. Cl. A61K 49/00; A61K 47/02; A61K 47/12; A61K 47/22; A61K 47/26; A61K 47/34

(52) U.S. Cl. CPC A61K 49/0043; A61K 47/02; A61K 47/12; A61K 47/26; A61K 2123/00

(57) ABSTRACT

Methods for early diagnosis of cervical intraepithelial neoplasia in low-income settings are disclosed. This invention primarily addresses inaccurate screening and diagnosis by developing, standardizing, and optimizing an innovative, highly-effective (high specificity, high sensitivity), and low-cost Fluorescein Sodium (FNa) staining method to replace the current burdensome (requires multiple visits), inherently inaccurate (low specificity, high sensitivity) Acetic Acid (AA) staining method.

### INCORPORATION BY REFERENCE OF ANY PRIORITY APPLICATIONS

Any and all applications for which a foreign or domestic priority claim is identified in the Application Data Sheet as filed with the present application are hereby incorporated by reference under 37 CFR 1.57. This application claims priority to U.S. Provisional Application No. 63/498,103, filed on Apr. 25, 2023, and is incorporated herein in its entirety by reference.

### BACKGROUND

The disclosed subject matter generally relates to optimization of a contrast agent solution and, more particularly, to the early diagnosis of cervical cancer by way of the administration of optimized fluorescein sodium formulation.

### SUMMARY

For purposes of summarizing, certain aspects, advantages, and novel features have been described herein. It is to be understood that not all such advantages may be achieved in accordance with any one particular embodiment. Thus, the disclosed subject matter may be embodied or carried out in a manner that achieves or optimizes one advantage or group of advantages without achieving all advantages as may be taught or suggested herein.

In some embodiments, a method of preparing an optimized contrast agent solution for diagnosing a cancer includes obtaining a plurality of contrast agent strips; adding the plurality of strips of contrast agent into a sterile normal saline solution, in a container; dissolving the plurality of strips of contrast agent in the saline solution to form a contrast agent solution by vigorously shaking the container; adjusting a pH of the contrast agent solution using citric acid and sodium citrate dihydrate; and adjusting a osmolality of the contrast agent solution using sodium chloride and potassium chloride.

In some embodiments, the optimized contrast agent solution is Fluorescein Sodium solution. In some embodiments, the mass of each of the plurality of the contrast agent strip is 1 mg. In some embodiments, the volume of the sterile normal saline solution is 5 cc.

In some embodiments, the method also includes adjusting a tonicity of the contrast agent solution. In some embodiments, the tonicity of the contrast agent solution is adjusted using salt and sugars. In some embodiments, the method also includes addition of specific agents known to promote the contrast agent solution uptake by cells. In some embodiments, the specific agents comprises at least one of a acetate, citrate, lactate, Tetronic 1107, arachidonic acid, or dihomo-gamma linolenic acid.

In some embodiments, the cancer is cervical cancer. In some embodiments, the contrast agent solution is transparent yellow.

Another embodiment is a method of applying an optimized contrast agent solution to a cancerous area which includes pre-rinsing the cancerous area with 10 ml of normal saline; applying 10 ml of the optimized contrast agent solution over the cancerous area; and post-rinse the cancerous area with 10 ml of normal saline. In some embodiments, the optimized contrast agent solution is Fluorescein Sodium solution. In some embodiments, the cancerous area is a cervix.

In some embodiments, the normal saline was applied using a cotton swab or plastic pipette. In some embodiments, the optimized contrast agent solution was applied using a cotton swab or plastic pipette.

The details of one or more variations of the subject matter described herein are set forth in the accompanying drawings and the description below. Other features and advantages of the subject matter described herein will be apparent from the description and drawings, and from the claims. The disclosed subject matter is not, however, limited to any particular embodiment disclosed.

### BRIEF DESCRIPTION OF THE DRAWINGS

The accompanying drawings, which are incorporated in and constitute a part of this specification, show certain aspects of the subject matter disclosed herein and, together with the description, help explain some of the principles associated with the disclosed implementations as provided below.

FIGS. 1a and 1b illustrate standard images of a cervix for AA and FNa staining respectively.

FIG. 2a illustrates FNa stained cervix with confirmed cervical cancer. Spots with fluorescence intensities in the range of 0 to 10 showing the range of FNa sensitivity. FIG. 2b illustrates AA stained cervix is provided from the same patient with confirmed cervical cancer.

FIG. 3 illustrates standard images comparing AA staining positive and FNa not staining and Low Grade (LG) by Pathology.

FIG. 4 illustrates standard images of AA staining differently than FNa and biopsy based on AA was Low Grade (LG) by Pathology. In the first pair of drawings, AA would lead to biopsy of the right upper quadrant of the cervix; whereas, FNa would lead to biopsy of the left upper quadrant.

FIGS. 5a and 5b illustrate standard images of cervix with AA Staining of Normal (N) and High Grade (HG) by Pathology respectively.

FIG. 6a illustrates standard images of cervix with FNa Staining of Low Grade (LG) by Pathology, selecting the most distinct of the available that is coincident with AA staining. FIG. 6b illustrates standard images of cervix with FNa Staining of High Grade (HG) or Cancer (CA) by Pathology, selecting the least distinct of the available that is coincident with AA staining.

FIG. 7 illustrates standard images of cervix showing comparisons between staining by AA and FNa for High Grade (HG) by pathology. Each horizontal pair of images is from the same patient.

FIG. 8 illustrates FNa staining of a cervix with carcinoma in situ (specimen from a cold-knife conization procedure).

FIG. 9 illustrates Sectioning of cervical excision specimens (CKC or LEEP).

### DETAILED DESCRIPTION

Unless otherwise defined, all technical and scientific terms used herein have the same meaning as commonly understood by one of ordinary skill in the art. Methods and materials are described herein for use in the present disclosure; other, suitable methods and materials known in the art can also be used. The materials, methods, and examples are illustrative only and not intended to be limiting. All publications, patent applications, patents, sequences, database entries, and other references mentioned herein are incorporated by reference in their entirety herein. In case of conflict, the present specification, including definitions, will control. Other features and advantages will be apparent from the following detailed description and figures, and from the claims.

For the purposes of this specification and appended claims, unless otherwise indicated, all numbers expressing quantities, percentages, or proportions, and other numerical values used in the specification and claims, are to be understood as being modified in all instances by the term “about,” to the extent they are not already so modified. Accordingly, unless indicated to the contrary, the numerical parameters set forth in the following specification and attached claims are approximations that may vary depending upon the desired properties sought to be obtained. At the very least, and not as an attempt to limit the application of the doctrine of equivalents to the scope of the claims, each numerical parameter should at least be construed in light of the number of reported significant digits and by applying ordinary rounding techniques.

It is noted that, as used in this specification and the appended claims, the singular forms “a,” “an,” and “the,” and any singular use of any word, include plural referents unless expressly and unequivocally limited to one referent. As used herein, the term “include” and its grammatical variants are intended to be non-limiting, such that recitation of items in a list is not to the exclusion of other like items that can be substituted or added to the listed items.

Cervical cancer (CC) is a cancer of economic, social, and educational disparities whose morbidity and mortality disproportionately affects vulnerable and disadvantaged populations. Women in rural areas are at higher risk for CC than in urban areas. A woman dies of CC every 2 minutes, with almost 0.6 million cases and 0.3 million deaths per year globally, despite CC being preventable and having screening guidelines. The World Health Organization (WHO) continues to bring low cost, simple, and comprehensive screening and treatment methods to low-to-middle-income countries (LMICs) or disadvantaged regions in developed nations, including rural areas. Over 85% of these cases occur worldwide in the LMICs or disadvantaged regions in developed nations mainly because the existing screening and diagnostic methods are inadequate. Even with the existing methods, the WHO call-to-action to eliminate CC by 2030 requires screening every woman over 35 years old at least once during her lifetime. In the USA, since the 1970s, a woman with an abnormal Pap smear typically undergoes 4 costly, time-consuming doctor's visits: screening (Pap smear), diagnosis (colposcopy), treatment, and follow up. Because of low diagnostic accuracy, regular screening is required.

Recently the WHO issued a “call to action” for the elimination of cervical cancer by 2030 (8). This call requires globally ensuring that 90% of women are HPV vaccinated, 70% of women are screened at least once between the ages of 35 to 45 years, and at least 90% of those detected with high grade (HG) lesions receive treatment (8). Vaccination alone is inadequate, and early detection is a key element in ensuring completion of the goal.

Many rural and underserved areas of the USA and the world have rates of cervical pre-cancer comparable to LMICs. The rural counties in Southwest Virginia (SW VA) have high rates (60.1% of diagnoses) of late-stage CC and are served primarily by mid-level providers. The problem is that care providers, especially mid-level providers, need an accurate (sensitive and specific), simple, low-technology, cost-effective, fast (same office visit), staining to identify HSIL and cervical cancer.

The currently available acetic acid (AA) mixtures for diagnosis of cervical cancer are typically compounded in the form of liquid and applied gently using a cotton swab that is dipped into a vial of AA and applied onto the affected area. The clinician needs to be confident, skilled, and meticulous in performing this procedure to ensure the AA is applied to only the desired areas since the effects of AA are transient: the cell rehydrates, the cytoplasm is restored, and any denatured species revert to their normal state. However, the premalignant and malignant cells have a higher nuclear-to-cytoplasmic ratio, so they reflect considerably greater amounts of white light than normal cells when AA is applied.

One major concern regarding the application of AA for diagnosis of cervical tissue is that the visual effect of AA is transient and requires repeated application. It has high false positive rates of 4% to 33% and high false-negative rates of 40% to 62% with high inter-observer variability. Most significantly, the specificity of AA to distinguish high grade (HG) lesions from low grade (LG) lesions remains low which indicates a substantial number of women will be undiagnosed or overtreated. Missed diagnosis leads to more frequent retesting, so the current standard of care recommends retesting every 3 to 5 years. Both missed diagnosis and overtreatment translate to greater morbidity and economic burden on the patient and the healthcare system.

Thus, there is a need for improving the specificity of colposcopy to accurately detect and distinguish HG lesions. A more specific contrast agent would be advantageous. Subtle color variations and low contrast with AA in colposcopy lead to interobserver variations; however, fluorescence or illuminance is a property that could eliminate this interobserver variation. Certain substances emit fluorescence when a particular wavelength of light is incident upon them. Therapeutic photodetection is based on the use of fluorescent dyes which, when illuminated with a particular wavelength of incident light, emit a light of longer wavelength (21).

Pap smear has been the traditional screening method for cervical cancer since the 1960s; however, due to an improved understanding of the pathogenesis of cervical cancer, pap smear is being replaced by HPV typing (11). Patients with abnormal pap smear or HPV typing results are provided “diagnosis” with colposcopy, a method for assessing the cervix under illumination and magnification. The current method for diagnosing cervical cancer is colposcopy during which AA is used as a staining agent to visualize abnormal/precancerous tissue. Colposcopy is based on discriminating the subtle discolorations of the cervical tissue surfaces. Colposcopic visualization is aided by the staining agents AA and Lugol's Iodine. Even with these staining agents, Colposcopy requires extensive expertise and training and is typically done by physicians (12). Colposcopy has contributed to reducing the morbidity and mortality of cervical cancer in the United States and many other parts of the world. However, the currently established diagnostic method of colposcopy with biopsy underestimates the disease severity in as many as 53% women with high grade squamous intraepithelial lesions (HSIL) in low resource settings (13). The ATHENA trial of over 47,000 women with abnormal cytology or HPV results in 2014 demonstrated that colposcopy missed 20.9% CIN 2 and 18.9% of CIN 3 in the USA (14). In conjunction with AA, the sensitivity of colposcopy to distinguish normal from abnormal tissue is relatively high, however, to distinguish low-grade lesions from high-grade lesions and cancer remains low indicating that a substantial number of women may be overtreated. “This analysis clearly demonstrates that colposcopy is far from perfect and likely misses clinically significant disease (14).”

The provider depends on visual perception for discriminating the suspicious lesions on the cervix and taking biopsies. Colposcopy adds magnification to the equation enhancing the ability to detect cervical intraepithelial neoplasia. Colposcopes provide magnification from 6 to 40-fold. Low and medium magnification are used for initial assessment, and high magnification (20-fold plus) is used to detect the finer detail of vascular patterns. In a meta-analysis, colposcopy was found to be sensitive (96%) for the detection of CIN2+ with a specificity of only 48%. (15).

The current diagnostic methods of patients with cervical intraepithelial neoplasia include the following diagnosis in order of tissue destruction and/or removal levels, starting with the least invasive and destructive method: Loop Electricosurgical Excision Procedure (LEEP) and Cold Knife Conization (CKC).

A better staining agent that can overcome these limitations is needed, which retains the low-cost and ease-of-use of AA. Attempts to improve the interpretation of AA staining with training, artificial intelligence (AI), and handheld colposcopes has not overcome the inherently low specificity of AA staining.

Fluorescein Sodium (FNa) formulations of the current disclosure can advantageously be used as the principal staining agent for the visual diagnosis of cervical precancer and overcome the limitations of existing diagnostic methods. FNa used to identify oral and other cancers intravenously, cervical cancer screening requires a topical formulation.

The FNa formulations and methods described herein provide specific, high contrast, sharp-edged identification specific to cervical cancer and improve diagnosis with higher accuracy than current staining agents used in colposcopy.

The devices described herein are also designed to facilitate image capture and improving contrast. In some embodiments, disclosed FNa formulation may be used within a Convenience Kit for colposcopy; in a kit converted to Visual Inspection with Fluorescein Sodium (VIF); and/or with complementary speculum improvements which enable early diagnosis of high grade lesions in the cervix in the human body.

As used herein, the term “cancer” has its ordinary meaning as understood in light of the specification, and refers a malignant tumor of potentially unlimited growth that expands locally by invasion and systemically by metastasis. A cancer may include, but is not limited to, cervical cancer, Bladder cancer, Breast cancer, Colorectal cancer, Kidney cancer, Lung cancer, Non-Hodgkin lymphoma, Melanoma, Oral and oropharyngeal cancer, Pancreatic cancer, and Prostate cancer.

In the following, numerous specific details are set forth to provide a thorough description of various embodiments. Certain embodiments may be practiced without these specific details or with some variations in detail. In some instances, certain features are described in less detail so as not to obscure other aspects. The level of detail associated with each of the elements or features should not be construed to qualify the novelty or importance of one feature over the others.

Improvements and optimization of an FNa formulation that is to be applied to the cervix during colposcopy are provided. In particular, the formulation of the composition is optimized for diagnosis of cervical intraepithelial neoplasia (CIN) 2. Further, the current disclosure relates to devices for using the composition, and combinations of composition and devices which facilitate the use of these formulation.

Medical devices and methods for optimization of topical formulation of physiologically acceptable liquids, gels, or dissolvable films for early diagnosis of cervical intraepithelial neoplasia are described. The formulation can be optimized for early and highly specific diagnosis of high grade and low grade cervical lesions and compared with the sensitivity and specificity of using AA on the cervix in the body during colposcopy.

Cervical cancer occurs at the squamous epithelium of the cervix, particularly at the squamo-columnar junction of the ecto- and endocervix, a site of continuous metaplastic activity. Persistent infection of the cervical epithelium with one or more oncogenic types of HPV leads to precancerous lesions, which, if not diagnosed and treated, gradually progress to invasive cervical carcinoma within a period of 10-20 years.

FNa may be used to visually detect cervical cancer by the method of Visual Inspection with Fluorescein Sodium (VIF). VIF capitalizes on the benefits (low cost, ease-of-use, simplicity, safety) of VIA while correcting the deficiencies (subjectivity, variability, difficulty of interpretation, inaccuracy, and nonspecificity). Some innovations described herein include: 1) replacing VIA with VIF for screening; 2) using VIF for diagnosis; 3) enabling single visit “diagnose and treat;” 4) using FNa to guide colposcopy and biopsy; 5) facilitating the use of AI currently being developed for AA. FNa fluorescence images are more localized, sharper, and clearer than AA images. The higher selectivity and specificity of VIF will combine screening and diagnosis into a single diagnostic visit with a mid-level practitioner, and this efficiency will improve precancer screening efficacy and clinical outcomes, that is, save women's lives.

Furthermore, FNa staining has several other advantages over conventional AA staining, while preserving AA staining's advantages. When tested, staining with FNa had a longer duration (more than 15 minutes) than AA (less than 1 minute) which enables better visualization and discrimination. Staining with FNa did not cause the same burning sensation that AA causes. The FNa staining was strong enough to not require a colposcope. In some embodiments, FNa staining may be observed with an unaided eye. Using FNa as a staining agent enables use in low resource settings where access to a colposcope is not possible. The FNa staining had more dramatic contrast which might lead to more precise biopsies and might aid in more precise excision in LEEP and CKC. FNa agent stating is usable to guide LEEP and CKC. FNa can preferentially stain high grade margins and not include low grade margins. FNa can spare tissue sparing compared to AA. In tumor surgeries, the margins delineated by FNA staining are accurate.

The present disclosure is based, in part, on the optimization of Fluorescein (3′,6′-dihydrospiro[isobenzofuran-1(3H),9′-[9H] xanthene]-3-one) sodium (“fluorescein”) which is a yellow, relatively nontoxic, hydroxyxanthene dye that produces an intense green fluorescence in slightly acid to alkaline (pH>5) solutions. It is an organic dye which has high quantum yield.

The mechanism for FNa accumulation is that the extracellular fluid in malignant tumors is more acidic than in the normal tissues, and the pH inside a tumor cell is neutral or slightly alkaline. FNa dye results in fluorescein accumulation inside cancer cells, which is governed by the pH gradient between the cell and the extracellular fluid, with an increased gradient present in tumors as compared to normal tissue. The extracellular fluid in malignant tumors is more acidic than in normal tissue, thus the FNa accumulation in tumors in vivo is governed by the pH gradient between the cells and the extracellular fluid. Research demonstrated that fluorescein sodium is preferentially retained in malignant tissue compared to normal tissue. The microscopic examination of fluorescein-stained tumor cells has revealed that the tumor uptake is 23-fold higher than in the surrounding tissue (53). Hematoxylin/eosin staining of the borders of the fluorescein-stained tumor cells has been shown to correspond exactly with the fluorescein borders (53).

Embodiments of the methods provided herein provide additional advantages, including, for example, the FNa signal-to-noise ratio is maximized by optimizing the FNa formulation, illumination source, and optical filters on patients that already have cervical cancer. Specifically, maximizing relative illumination (intensity) and discrimination (contrast at margins) of abnormal and normal tissue optimizes the formulation for pH, tonicity, composition, and concentrations of FNa and other ingredients through testing on recruited subjects scheduled to have LEEP or CKC treatment. In some embodiments, the optical filters for the illumination source and the observer's eyes may be optimized in tandem with the formulation.

In addition, FNa distinguishes between high grade and low grade lesions with higher specificity than AA as shown by using p16, Ki67, and c-myc FISH staining of specimens. This indicates that FNa stains only high grade CIN 2 and worse and AA is less specifically targeted and stains any lesion from CIN 1 and worse. Thus, FNa's higher specificity reduces the rate of false positive results.

Existing similar technologies demonstrated the potential discriminating power of FNa and highlighted areas for improvement in the unoptimized formulation and technique—intensity, contrast, and consistency. The present disclosure enables optimization of metrics for intensity (relative illumination) and contrast at margins (discrimination between normal and abnormal tissues), standardization of the clinical technique, and optimization of the formulation composition, light source, and optical filters to improve the metrics of intensity and contrast by using an on-cervix formulation-comparison approach using clinical CKC and LEEP patients and the subsequent pathology slicing.

The pH of the FNa delivery solution is optimized to ensure consistency of cervical pH for FNa uptake. The FNa delivery solution is pH and tonicity adjusted to promote both passive transport (unprotonated) and active transport (monoanionic) as observed in human intestinal cells, and supplemented with specific agents known to promote FNa uptake by cells, including acetate or citrate, and lactate. Other agents that promote cellular uptake in general, such as Tetronic 1107, glucose and arachidonic acid or dihomo-gamma linolenic acid may be incorporated. The formulation development steps proceed through pH selection (buffers), then tonicity (salts, sugars), then additional ingredient identification, and finally concentration optimization. Specifically, after selecting pH, tonicity, and ionic strength, a formulation may include one or more of acetate, citrate, lactate, Tetronic 1107, arachidonic acid, and dihomo-gamma linolenic acid. Other permeation enhancers may be incorporated.

In some embodiments, FNa may be present in pure form or up to 50% by mass in the formulation. The pH of the delivery solution may be between 2.0 and 10.0 such as 2.0, 2.5, 3.0, 3.5, 4.0, 4.5, 5.0, 5.5, 6.0, 6.5, 7.0, 7.5, 8.0, 8.5, 9.0, 9.5 or 10.0, or an amount within a range defined by any two of the aforementioned values. The tonicity of the formulation may be within up to 2 orders of magnitude from that of normal blood plasma.

Acetate, citrate, lactate, Tetronic 1107, chlorides, phosphates, other simple salts, glucose, sucrose, fructose, other sugars and simple carbohydrates, ethyl alcohol and other simple alcohols, arachidonic acid, dihomo-gamma linolenic acid, and other permeability enhancers may be present in the formulation at safe concentrations up to their solubility limits or 15% by weight, whichever is higher.

The formulation may include a mixture of chemicals including FNa in concentrations of 0.05 mg/ml to 0.5 mg/ml, depending on intended use, such as 0.05, 0.06, 0.07, 0.08, 0.09, 0.1, 0.15, 0.2, 0.25, 0.3, 0.35, 0.4, 0.45, or 0.5 mg/ml, or an amount within a range defined by any two of the aforementioned values.

The present disclosure provides examples of LED light sources (for example, Elipar™ FreeLight 2 LED Curing Light (3M); Elipar™ Optilux™ 501 Curing Light; and commonly available LED flashlights, such as Maglite or Olight) may be used (images captured and documented). After selection of the blue LED light source based on intensity and contrast observed, the same light source is used for all subsequent measurements.

In one aspect, for example, the Elipar™ series by 3M provides suitable light, and has been used in dentistry for many years. The absorption spectrum for FNa ranges from 400 nm to above 500 nm, and becomes more peaked near 494 nm with higher pH. Both blue and green LEDs, as typically found in flashlights, encompass the target source wavelength of 494 nm.

The present disclosure also provides that the use of optical filters accentuates contrast. The optical filters on the source remove wavelengths in the emission range (525 nm), and similarly, the filters on the viewer remove wavelengths in the source range (494 nm). For example, blue-light-filtering clip-on lenses may be applied. In another aspect, for example, Thor Labs MDF-GFP2 optical filter sets are designed for Fluorescein type fluorophores. The same optical filters used for the observer will be imposed on the camera for capturing images.

In one aspect, for example, Image analysis software, MATLAB with the Medical Imaging Toolbox and Image Processing Toolbox, may be used to accurately characterize the intensity at each point and the margin contrast at each boundary between fluorescing (green) and non-fluorescing (blue) regions. This software is used for similar image processing of AA stained cervixes to identify regions of interest (ROI).

### EXAMPLES

Some aspects of the embodiments discussed above are disclosed in further detail in the following examples, which are not in any way intended to limit the scope of the present disclosure. Those in the art will appreciate that many other embodiments also fall within the scope of the disclosure, as it is described herein above and in the claims.

### Example 1

The following example demonstrates a method to optimize a contrast agent solution.

The FNa contrast agent solution was prepared by dissolving three (3) ophthalmic FNa strips. Each strip containing 1 mg of FNa, was dissolved in 5 cc of sterile normal saline, resulting in a transparent yellow dye. The FNa concentration of the contrast agent solution was adjusted to 0.3 mg/mL FNa and pH was adjusted to a 5.1 with citric acid (1 g/l) and sodium citrate dihydrate (2.5 g/l). The osmolality was adjusted with 275 mOsmol sodium chloride and 25 mOsmol potassium chloride, and containing one (1) g/L of glucose. In some embodiments, a diagnostic kit can be prepared where the solution described above, except for the FNa, is premixed and the FNa is added just prior to use. Dissolution of the FNa in the solution prior to use will be conducted through vigorous shaking of the vial for at least 10 seconds or until no visible solids are seen, then gentle motion of the fluid, at least 60 seconds.

### Example 2

The following example demonstrates a method of applying a FNa contrast agent solution, such as that described in Example 1, to the cancerous area in the cervix.

The FNa formulation is applied in 3 steps, each using either a cotton swab or plastic pipette for delivery and continuing until all solution is dispensed: a) Pre-rinse with 10 mL of normal saline to remove cervical mucus. b) Apply 10 mL of a FNa delivery solution over the cervix. Solution in the posterior cul-de-sac is picked up with the pipette and resprayed, at least once, then discarded. c) after imaging or diagnosis, post-rinse with 10 mL of normal saline.

### Example 3

The following example describes a method for comparing the staining of the abnormal tissues and that FNa has a higher specificity than AA with comparable sensitivity. Each patient underwent standard colposcopy, which was performed consecutively with AA and then FNa as contrast agents.

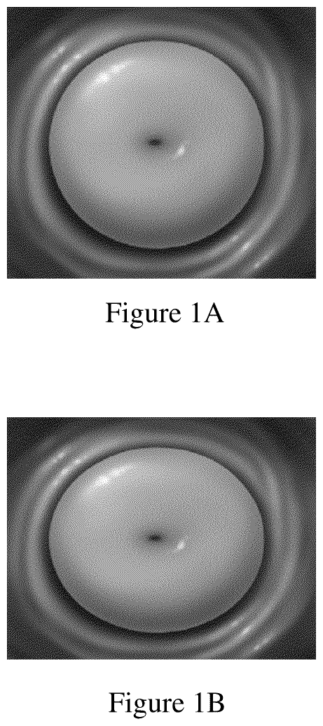

The observer first applied AA to the cervix. FIG. 1A illustrates AA staining of normal tissue as documented on a standard diagram of the cervix. The patient's cervix was then rinsed with normal saline and FNa formulation was applied by the same colposcopist, and the FNa staining of normal tissue was documented on a separate standard diagram of the cervix as shown in FIG. 1B. FIGS. 1A-B indicate that there are no cancer cells detected.

### Example 4

The following example demonstrates that different levels of fluorescence in a FNa cervical staining shows different grade of cancer.

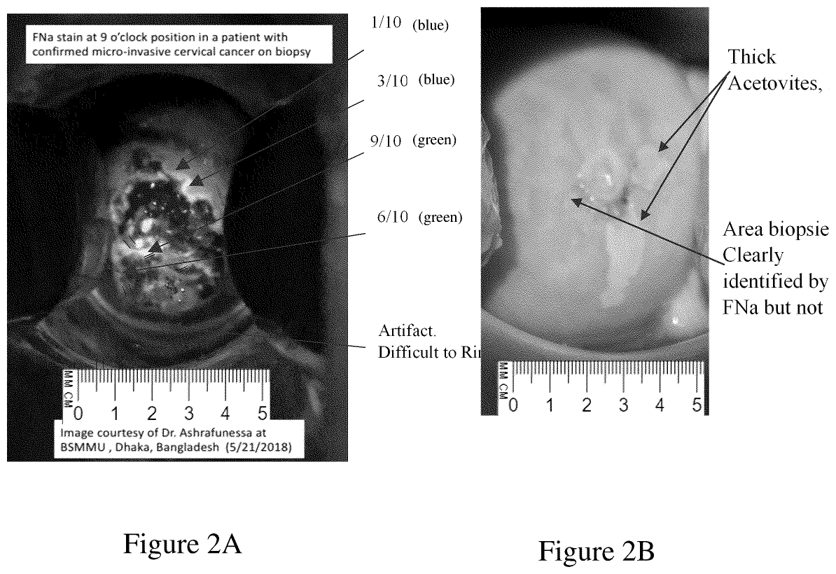

FIG. 2A illustrates an example of cervical FNa staining displaying different levels of fluorescence (green) intensity and the variation in the size of the margins between high grade (green) and other tissues (blue).

The regions in FIG. 2A have been manually labeled with a numeric intensity from the lowest 0/10 (blue) to the highest 10/10 (green) and bars spaced 0.5 mm apart and 2 mm apart have been placed to identify some margins. The clinician-desired-target margin is 0.5 mm, for that is the approximate precision of biopsy targeting, which is conservative compared to the standard deviation in endoscopic forceps cervical biopsy diameter of 0.8 mm. A manual estimate of the average margin depicted is 2 mm. The approximate variation (standard deviation) in margin size is 1 mm. Using a standard deviation of 1 mm, a detectable difference of 2 mm−0.5 mm=1.5 mm, a type I (alpha) error of 5% (confidence of 95%), and a type II (beta) error of 10% (power of 90%), the required number of samples for each pair of formulations is 7 using SAS Institute JMP© 17 statistical software for a one-sample mean-paired-difference vs. zero.

Applying different formulations to the same cervix on multiple visits before the CKC or LEEP procedure keeps the disease the same between two or more formulations. Selection decisions can be made after each set of 7 patients. Combining the photographs of the cervix with FNa (FIG. 2A) or AA (for example, FIG. 2B shows the same cervix pictured after AA staining) and pathology results, the different formulations will be compared.

### Example 5

The following example demonstrates that a diagnostic performance was evaluated for AA and FNa staining using histopathology as the reference diagnosis in patients with pap smear abnormalities.

For the detection of low grade SIL or worse per histopathology, AA staining demonstrated 99% sensitivity, 43% specificity, 95% positive predictive value (PPV), and 75% negative predictive value (NPV). FNa staining demonstrated 94% sensitivity, 86% specificity, 99% PPV, and 60% NPV. FNa had a higher diagnostic specificity than AA (86% vs. 43%). The only differences in diagnosis between AA and FNa staining occurred in patients with low grade SIL per histopathology, with AA having three more false positives than FNa and FNa having three more false negatives than AA. For the diagnosis of high grade SIL or worse per histopathology, both AA and FNa had 94% sensitivity; whereas, FNa had 86% specificity for high grade SIL as compared to AA which had only 43% specificity. This is shown below in Tables 1 and 2.

TABLE 1
Correlation of FNa staining with biopsy report.

| Test: FNa | Ca, HG, LG | BG, N | Total |
|---|---|---|---|
| FNa Positive | 67 | 1 | 68 |
| FNa Negative | 4 | 6 | 10 |
| Total | 71 | 7 | Sensitivity = 94% |
| | | | Specificity = 86% |
| | | | PPV = 99% |
| | | | NPV = 60% |
| | | | Accuracy = 94% |

TABLE 2
Correlation of AA staining with biopsy report.

| Test: AA | Ca, HG, LG | BG, N | Total |
|---|---|---|---|
| AA Positive | 70 | 4 | 74 |
| AA Negative | 1 | 3 | 4 |
| Total | 71 | 7 | Sensitivity = 99% |
| | | | Specificity = 43% |
| | | | PPV = 95% |
| | | | NPV = 75% |
| | | | Accuracy = 94% |

The results clearly show that FNa composition of the current disclosure has a higher specificity than AA with comparable sensitivity. Neither AA nor FNa missed any high grade lesions; however, biopsy guided by AA resulted in more normal or low grade lesions which correlates with the high cost associated with AA colposcopy.

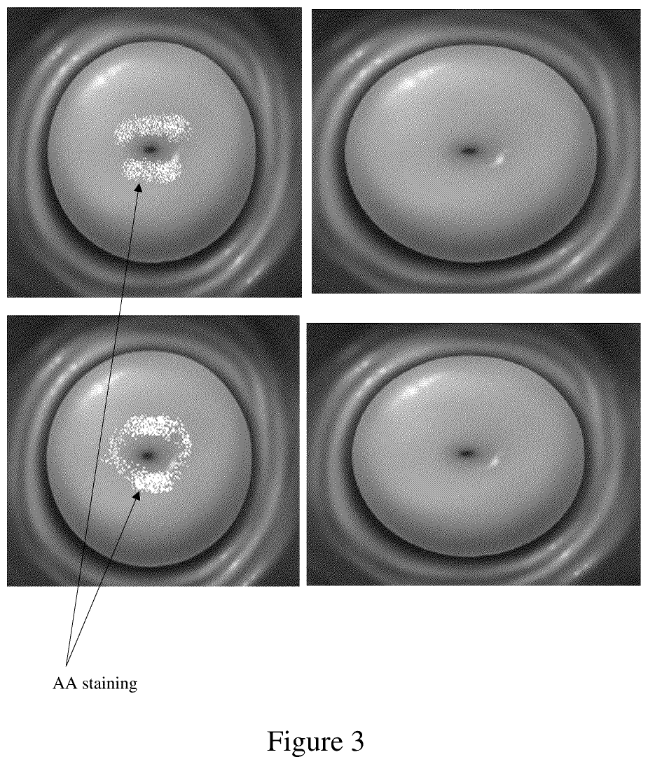

In one case, Cytology, AA, and FNa all were positive, but Pathology was negative. In four cases, cytology and AA were positive, FNa was negative, and the pathology indicated only low grade cancer cells, which illustrates that diagnosis relying on cytology and AA would lead to overtreatment. In FIG. 3, the left two quadrants show a diagram of a cervix stained with AA, resulting in a positive indication of low grade cancer cells. The right two quadrants show a diagram of the same cervix stained with FNa, showing a negative result for the same low grade cells. Because the pathology was low grade, using AA would result in a false positive and could lead to unnecessary or overtreatment. Using FNa compositions of the current disclosure results in fewer false positives than using AA.

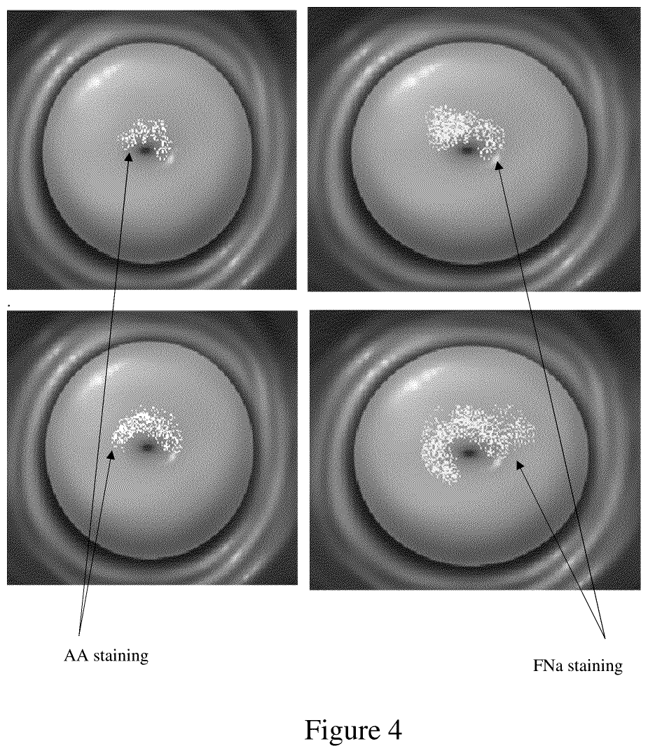

Referring to FIG. 4, AA stains a different region than FNa of a cervix, and biopsy based on only AA showed LG, posing the possibility of missing biopsy of the location of the HG. Biopsy based on AA did not capture the additional areas stained by FNa that may have had different pathology. The top left quadrant shows AA positive staining and the top left quadrant shows FNa positive staining for low grade. The extent of the AA positive staining would lead to biopsy of the right upper portion of the cervix, and FNa positive staining would lead to biopsy of the left upper portion on the same cervix. The bottom two quadrants shows AA positive staining on the right and FNA positive staining on the left for low grade. The AA positive staining would lead to biopsy of the left upper portion of the cervix and FNa positive staining would lead to biopsy at 12 o'clock on the same cervix. By taking the biopsy in the areas indicated by the AA staining, HG cells could have been missed, which would result in a failure to identify or diagnose the HG cells, and potential undertreatment.

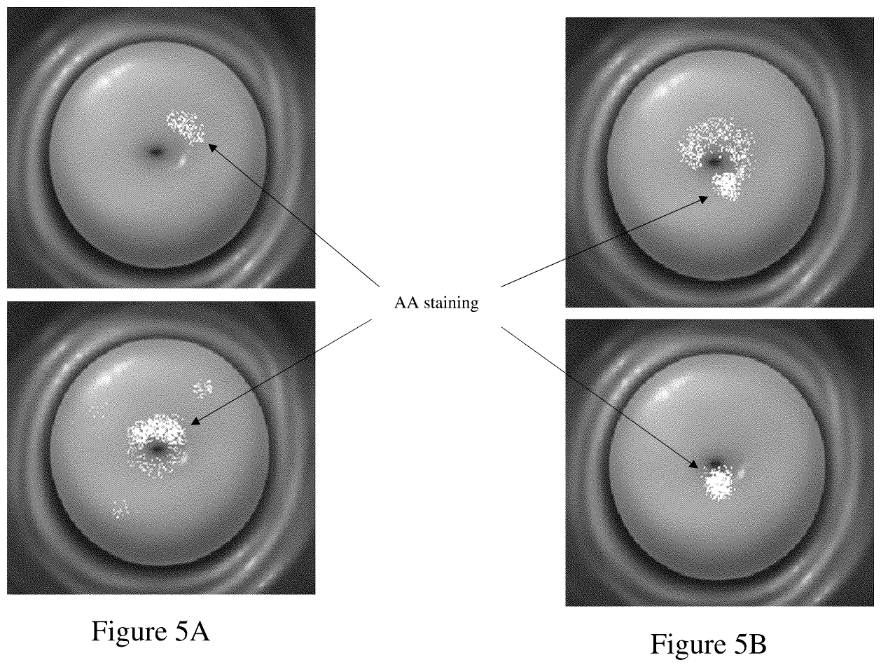

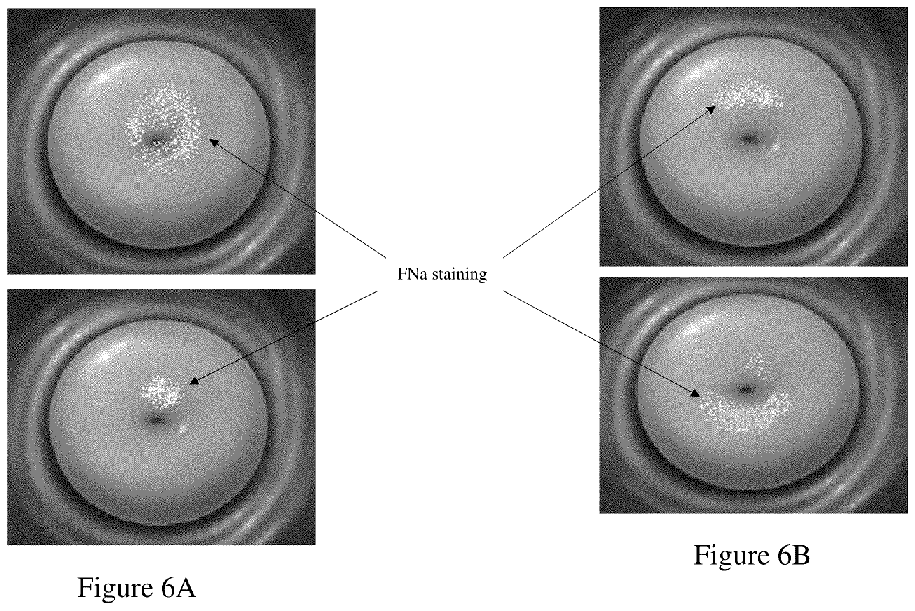

Referring to FIGS. 5A and 5B, normal and HG pathology are often indistinguishable by AA staining. FIG. 5A shows AA positive staining for normal tissue according to pathology. FIG. 5B shows AA positive staining for the same high grade according to pathology. For a conservative comparison, FIG. 6A illustrates that the LG by pathology that was most distinctly stained by FNa is still distinguishable from the HG by pathology that was least distinctly stained by FNa as illustrated in FIG. 6B. The distinguishability between LG and HG for FNa can be highlighted based on the FNa formulations described herein.

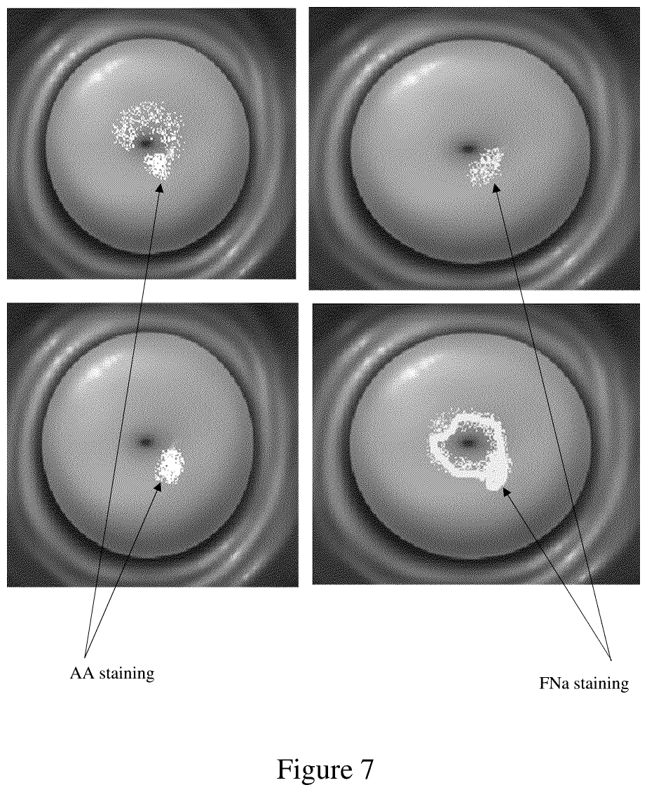

FIG. 7 illustrates the comparison between AA and FNa staining for cases that were HG by pathology. The top two quadrant shows AA positive staining on the right and FNA positive staining on the left for high grade on the same cervix. The bottom two quadrant shows AA positive staining on the right and FNA positive staining on the left for high grade, also on the same cervix.

LG and HG stains by AA often look the same. Since AA is non-specific mechanistically, changing AA concentrations or formulations cannot distinguish between LG and HG cells.

Based on the preference that FNa seems to have for high grade lesions, it can be hypothesized that FNA discriminates between CIN2+ lesions that will develop into cancer and CIN2− lesions which will regress. If the hypothesis is correct, then FNa would better match with the desired standard of care for evaluation of lesions. That is, the standard of care to “wait and see” for low grade lesions aligns with the staining of FNa, and the standard of care only to treat high grade lesions aligns with the staining of FNa.

The Table 3 below is an example of the comparison of FNa staining with pap smear, AA and biopsy.

TABLE 3
Comparison of Flouroscein sodium staining results
with pap-smear, acetic acid and biopsy.

| | Acetic | | | High grade | Low grade | | Benign |
|---|---|---|---|---|---|---|---|
| | acid | Flouroscein | | biopsy | Biopsy | Invasive | biopsy |
| Pap smear | positive | sodium | | (n = 26) | (n = 41) | (n = 5) | n = 6 |
| High grade | 20 | Positive | 20 | 12 | 5 | 3 | 0 |
| (n = 22) | | Negative | 2 | 0 | 0 | 0 | 2 |
| Low grade | 44 | Positive | 39 | 9 | 29 | 1 | 0 |
| (n = 46) | | Negative | 7 | 0 | 3 | 0 | 4 |
| Invasive | 5 | Positive | 5 | 4 | 0 | 1 | 0 |
| (n = 5) | | Negative | 0 | 0 | 0 | 0 | 0 |
| No pap | 5 | Positive | 4 | 1 | 3 | 0 | 0 |
| smear | | Negative | 1 | 0 | 1 | 0 | 0 |
| (n = 5) | | | | | | | |
| (pap report | | | | | | | |
| not | | | | | | | |
| available at | | | | | | | |
| time of | | | | | | | |
| colposcopy) | | | | | | | |

### Example 6

The following example demonstrates that on-cervix formulation-comparison analysis approach before carrying out LEEP or CKC.

Studies compared clinical screening of different women (with or without CIN 2 or worse) to compare sensitivity and specificity between AA and FNa. To optimize the FNa formulation, a radically different approach focuses only on women who have been already diagnosed with CIN 2 or worse. In the majority of these cases, not all the cervix is impacted; nevertheless, the current standard of care with LEEP or CKC is to remove exhaustively all sections of the cervix that might have cancer, which includes regions of various degrees of abnormal tissue.

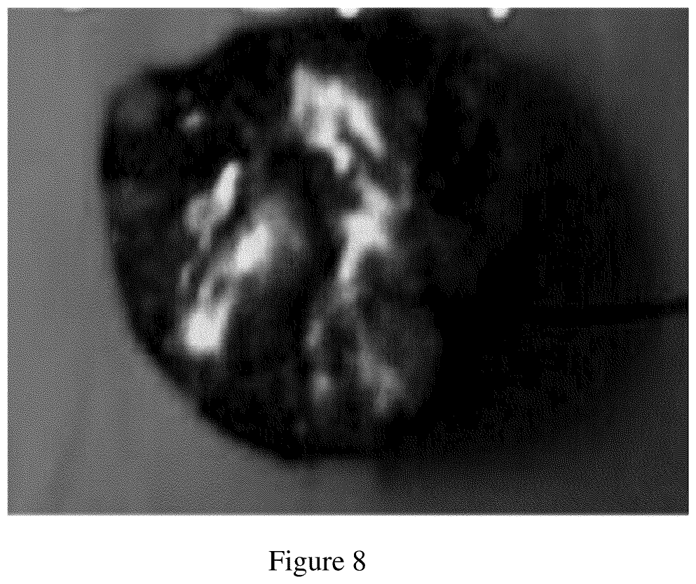

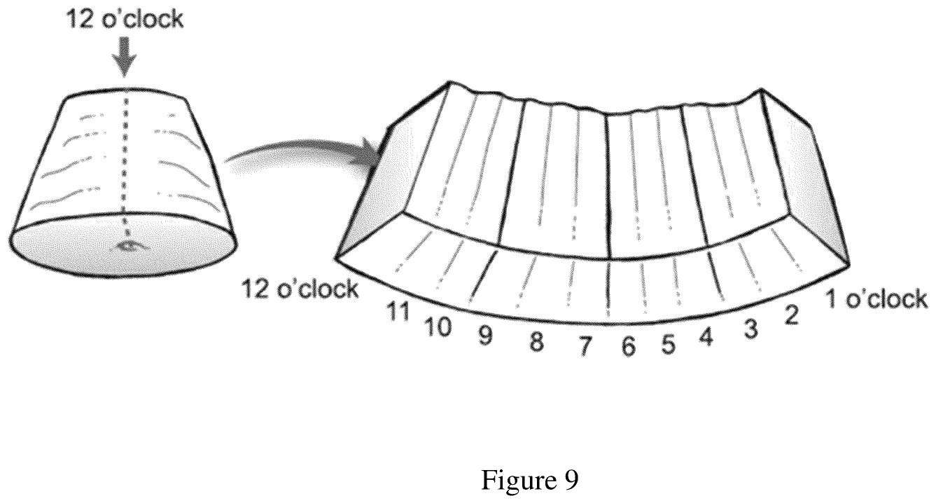

In FIG. 8, an example of a CKC specimen with FNa staining identifying regions of cancer is shown. Pathological analysis then dissects the removed tissue as shown in FIG. 9 to identify the extent of Cervical Intraepithelial Neoplasia (CIN) in each of the dissection sections using standard pathology grading. Because the sections are marked (similarly to the dial on an analog clock), the CIN levels of each section can be mapped back to the AA and FNa stained pictures of the same cervix taken before the LEEP or CKC procedure. This comparison provides a highly efficient means to optimize formulations and compare analytical (not clinical) sensitivity and specificity, that is, the capability of each of the different stains to identify tissue compared to pathology.

It is also understood that throughout the application, data is provided in a number of different formats, and that this data, may represent endpoints or starting points, and ranges for any combination of the data points. For example, if a particular data point “10” and a particular data point “15” may be disclosed, it is understood that greater than, greater than or equal to, less than, less than or equal to, and equal to 10 and 15 may be considered disclosed as well as between 10 and 15. It is also understood that each unit between two particular units may be also disclosed. For example, if 10 and 15 may be disclosed, then 11, 12, 13, and 14 may be also disclosed.

In accordance with certain embodiments, the methods are particularly implemented to be suitable for use in low-to-middle-income countries, where access to medical treatment is difficult, due to the complexity of the treatment options and the respective costs. Through this method, we aim to popularize a more accurate screening and affordable diagnosis in low-to-middle-income countries where over 85% of the cervical cancer diagnoses occur.

In this application, numerous specific details are given to provide a thorough understanding of the invention. However, it will be apparent that the invention may be practiced without these specific details. In order to avoid obscuring the present invention, some well-known compositions, system configurations, and process steps are not disclosed in detail. The drawings showing embodiments of the system are semi-diagrammatic and not to scale and, particularly, some of the dimensions are for the clarity of presentation and are shown exaggerated in the drawing figures.

Where multiple embodiments are disclosed and described having some features in common, for clarity and ease of illustration, description, and comprehension thereof, similar and like features one to another will ordinarily be described with similar reference numerals. Although various illustrative embodiments have been disclosed, any of a number of changes may be made to various embodiments without departing from the disclosures herein.

For example, the order in which various described method steps are performed may be changed or reconfigured in different or alternative embodiments, and in other embodiments, one or more method steps may be skipped altogether. Optional or desirable features of various device and system embodiments may be included in some embodiments and not in others. Therefore, the foregoing description is provided primarily for the purpose of example and should not be interpreted to limit the scope of the claims and specific embodiments or particular details or features disclosed.

The examples and illustrations included herein show, by way of illustration and not of limitation, specific embodiments in which the disclosed subject matter may be practiced. As mentioned, other embodiments may be utilized and derived therefrom, such that structural and logical substitutions and changes may be made without departing from the scope of this disclosure. Such embodiments of the disclosed subject matter may be referred to herein individually or collectively by the term “invention” merely for convenience and without intending to voluntarily limit the scope of this application to any single invention or inventive concept, if more than one is, in fact, disclosed. Thus, although specific embodiments have been illustrated and described herein, any arrangement calculated to achieve an intended, practical or disclosed purpose, whether explicitly stated or implied, may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

The disclosed subject matter has been provided here with reference to one or more features or embodiments. Those skilled in the art will recognize and appreciate that, despite of the detailed nature of the example embodiments provided here, changes and modifications may be applied to said embodiments without limiting or departing from the generally intended scope. These and various other adaptations and combinations of the embodiments provided here are within the scope of the disclosed subject matter as defined by the disclosed elements and features and their full set of equivalents.

A portion of the disclosure of this patent document may contain material, which is subject to copyright protection. The owner has no objection to facsimile reproduction by any one of the patent documents or the patent disclosure, as it appears in the Patent and Trademark Office patent files or records, but reserves all copyrights whatsoever. Certain marks referenced herein may be common law or registered trademarks of the applicant, the assignee or third parties affiliated or unaffiliated with the applicant or the assignee. Use of these marks is for providing an enabling disclosure by way of example and shall not be construed to exclusively limit the scope of the disclosed subject matter to material associated with such marks.

1. A method of preparing an optimized contrast agent solution for diagnosing a cancer, the method comprising:
obtaining a plurality of contrast agent strips;
adding the plurality of strips of contrast agent into a sterile normal saline solution, in a container;
dissolving the plurality of strips of contrast agent in the saline solution to form a contrast agent solution by vigorously shaking the container;
adjusting a pH of the contrast agent solution using citric acid and sodium citrate dihydrate; and
adjusting a osmolality of the contrast agent solution using sodium chloride and potassium chloride.

2. The method of claim 1, wherein the optimized contrast agent solution is Fluorescein Sodium solution.

3. The method of claim 1, wherein the mass of each of the plurality of the contrast agent strip is 1 mg.

4. The method of claim 1, wherein the volume of the sterile normal saline solution is 5 cc.

5. The method of claim 1, further comprising adjusting a tonicity of the contrast agent solution.

6. The method of claim 5, wherein the a tonicity of the contrast agent solution is adjusted using salt and sugars.

7. The method of claim 1, further comprising addition of specific agents known to promote the contrast agent solution uptake by cells.

8. The method of claim 7, wherein the specific agents comprises at least one of a acetate, citrate, lactate, Tetronic 1107, arachidonic acid, or dihomo-gamma linolenic acid.

9. The method of 1, wherein the cancer is cervical cancer.

10. The method of claim 1, wherein the contrast agent solution is transparent yellow.

11. A method of applying an optimized contrast agent solution to a cancerous area, the method comprising:
pre-rinsing the cancerous area with 10 ml of normal saline;
applying 10 ml of the optimized contrast agent solution over the cancerous area; and
post-rinse the cancerous area with 10 ml of normal saline.

12. The method of claim 11, wherein the optimized contrast agent solution is Fluorescein Sodium solution.

13. The method of 11, wherein the cancerous area is a cervix.

14. The method of claim 11, wherein the normal saline was applied using a cotton swab or plastic pipette.

15. The method of claim 11, wherein the optimized contrast agent solution was applied using a cotton swab or plastic pipette.
